(19) United States
(12) Patent Application Publication
(10) Pub. No.: US 2009/0007909 A1
(43) Pub. Date: Jan. 8, 2009

(54) Inhalation device

(76) Inventor: Silvio Carrico, Singen (DE)

Correspondence Address: FLYNN THIEL BOUTELL & TANIS, P.C., 2026 RAMBLING ROAD, KALAMAZOO, MI, 49008-1631, US

(21) Appl. No.: 11/906,065

(22) Filed: Sep. 28, 2007

(30) Foreign Application Priority Data
Sep. 28, 2006 (DE) 10 2006 047 668.9

(51) Int. Cl. A61M 15/00; A61M 11/00

(52) U.S. Cl. 128/203.15; 128/200.22; 128/200.14

(57) ABSTRACT

An inhalation device for a powdery medium includes a housing having at least one medium reservoir and an outlet channel, in which a first end of the outlet channel opens out in the area of the medium reservoir and a second end is designed to lie flush on a respiration opening of a user, such that the medium stored in the medium reservoir can be inhaled by the user breathing in. A respiration assistance device is provided which can be connected releasably to the housing and from which an air stream assisting the inhalation can be delivered to the housing by a manual actuation.

### FIELD OF THE INVENTION AND PRIOR ART

The invention relates to an inhalation device for an in particular powdery medium, with a housing having at least one medium reservoir and an outlet channel, in which a first end of the outlet channel opens out in the area of the medium reservoir and a second end is designed to lie flush on a respiration opening of a user, such that the medium stored in the medium reservoir can be inhaled by the user breathing in.

An inhalation device of this kind is known, for example, from EP 0957962 B1. The device disclosed therein is composed of a plastic housing with two medium reservoirs whose powdery content is dispensed by insertion of an attachment piece into a nostril of the user and subsequent inhalation through the attachment piece. The device is suitable in particular for media that are to be taken in by the nasal route and that contain a medicinal active substance combination.

A disadvantage of said device, and of similar devices from the prior art, is considered to be that very young and elderly users in particular may have problems in being able to perform the inhalation procedure as strongly and for as long as is necessary to inhale all of the medium.

### OBJECT AND SOLUTION

The object of the invention is to create an inhalation device of the type in question in which the dispensing procedure can be carried out successfully and completely even when unpracticed or physically restricted users, in particular young or elderly users, employ the inhalation device.

According to the invention, this object is achieved by an inhalation device of the type mentioned at the outset that has a respiration assistance device which can be connected releasably to the housing and from which an air stream assisting the inhalation can be delivered to the housing by means of a manual actuation.

The device according to the invention is a two-part system whose components can be assembled and disassembled by the user. One component, the housing of the inhalation device, can preferably also be used separately, such that the respiration assistance device need only be used by those users for whom a correct inhalation procedure would otherwise be impossible. The housing of the inhalation device can be designed in one part or in several parts and is preferably made from plastic, for example by injection molding. It contains the medium reservoir in which the medium is stored until a dispensing procedure takes place. Until the time the inhalation device is used, this medium reservoir is preferably sealed by a foil, which is at least partially removed or punctured only in the course of an activation for the dispensing procedure and which beforehand prevents accidental escape of the medium. The outlet channel provided on the housing is designed such that it can lie flush on the respiration opening of the user. In an inhalation device according to the invention for nasal application, it preferably has a round or oval cross section, whose diameter is adapted more or less to the size of a nostril of the target group in question.

The respiration assistance device can be connected to this housing of the inhalation device in such a way that air escaping through an outlet opening in the respiration assistance device can flow into the housing. The housing is designed in such a way that this air stream is delivered to the medium reservoir and the medium is thereby blown into the outlet channel and then dispensed. The air stream can be generated directly by the manual actuation, for example using a manually actuated piston pump. However, it may also be expedient to use a pump means which is fed from an energy accumulator, for example an electrically operated pump means, in which an indirect manual actuation takes place only in the form of an actuation of a switch. The manual actuation preferably takes place on the respiration assistance device itself or in cooperation with the respiration assistance device, for example by pressing together the contact area of respiration assistance device and housing. However, embodiments are also conceivable in which the actuation of the respiration assistance device takes place on the housing.

During the use of an inhalation device according to the invention, the inhalation performed by the user and the actuation of the respiration assistance device by the user are coordinated in time in such a way that the powdery medium is at the same time drawn into the outlet channel by the user breathing in and is blown through the respiration assistance device into the outlet channel. The inhalation procedure is therefore particularly reliable, and, in problematic circumstances, for example with unpracticed or physically restricted users or even with particularly large amounts of the medium, it is possible to ensure that the entire amount of the medium is inhaled completely.

In addition to the advantage concerning successful inhalation, even by problematic target groups, an inhalation device according to the invention has further advantages too. The respiration assistance device can be used several times, which is of advantage from the point of view of protection of the environment. Moreover, when suitably configured, the housing can be used with and without the respiration assistance device without any special adaptation, with the result that, compared to alternative embodiments in which the respiration assistance device is an integral part of the inhalation device, larger and therefore more economic batches of the housing can be produced.

In a development of the invention, the respiration assistance device is designed for a manual actuation in a direction of actuation which is parallel to a main direction of orientation of the outlet channel.

This configuration avoids the danger where, in the course of manual actuation of the respiration assistance device, the outlet channel slips out of or away from the respiration opening of the user. An actuation leads at most to a movement in the main direction of orientation of the outlet channel, and in the case of nasal application thus has the effect that the outlet channel is pressed slightly farther into the nostril of the user.

In a development of the invention, the respiration assistance device has a compression chamber whose volume can be mechanically reduced directly by the manual actuation.

Such a respiration assistance device can be produced particularly simply and is therefore particularly inexpensive. In the simplest case, the respiration assistance device comprises a simple pump ball which, for actuation, is enclosed fully in the hand and then compressed.

It is particularly advantageous if the respiration assistance device has a bellows.

Because of the defined direction of actuation arising from the design, such a bellows forming the compression chamber can be operated with just one or two fingers, such that the entire inhalation device can be manipulated using just one hand. Such a bellows is preferably made from an elastomer or a thermoplastic, for example polyethylene. The bellows is preferably designed integrally with an adjoining connecting portion for securing it to the housing, the elastic material being advantageous when securing the respiration assistance device on the housing. However, other embodiments are also expedient in which the connecting portion is provided as a separate structural part which, for example, is connected to the bellows by form-fit engagement of a circumferential web of the bellows into a circumferential groove of the connecting portion. The orientation of the bellows is preferably such that a bellows compression takes place in the direction of the housing of the respiration assistance device. However, other embodiments are also conceivable, for example ones in which the bellows is actuated by pressing its two end faces together, in which case the housing is provided on an intermediate side of the bellows. The bellows is preferably produced by injection blow molding or injection stretch blow molding.

In a development of the invention, a finger support is provided on a side of the housing directed away from the respiration assistance device.

This finger support ensures reliable handling, in which the actuation of the inhalation device, in particular when using a respiration assistance device with a defined direction of actuation, is effected by simply pressing together the fingers engaged on opposite sides of the inhalation device.

In a development of the invention, the respiration assistance device and the housing are designed in a contact area in such a way that a force applied by the manual actuation leads to portions arranged on the housing and on the respiration assistance device being pressed tightly onto each other in the contact area.

The contact area can be formed, for example, by a circumferential annular surface on the housing of the inhalation device and a corresponding circumferential surface on the respiration assistance device. If, for actuation purposes, a force is applied with which the housing and the respiration assistance device are pressed onto each other, this leads to a particularly secure bearing of the surfaces on each other. Escape of relevant amounts of the air flowing out of the respiration assistance device into the environment is thus suppressed.

In a development of the invention, in a contact area, the respiration assistance device and the housing have, sealing and/or securing means acting with a force fit and/or form fit.

These sealing and/or securing means ensure a reliable connection of the housing of the inhalation device to the respiration assistance device. They ensure that, in the event of an overpressure in the respiration assistance device or elastic deformation of the respiration assistance device in the course of actuation, the latter does not come loose from the housing. The sealing and/or securing means can in particular be form-fit means such as locking arrangements, for example a locking lip arranged on the housing and engaging in a groove arranged on the respiration assistance device.

In a development of the invention, the securing means comprise a push-on cone on the housing and a push-on portion on the respiration assistance device.

Such a securing means has a force-fit action. The push-on portion is pushed onto the push-on cone until a sufficient retaining force is achieved by a resulting elastic expansion of the push-on portion and elastic compression of the push-on cone. Embodiments are particularly preferred in which an air channel for transporting air into the housing remains open in the contact area, for example through axial grooves on the cone or through tensioning ribs formed internally on the push-on portion and which, in the secured state, bear on the push-on cone, while areas between the tensioning ribs are spaced apart from the push-on cone.

In a development of the invention, the housing is designed in several parts, in particular in two parts, the at least one medium reservoir being designed in a first housing part, and the outlet channel being designed on a second housing part, and a circumferential contact area between the respiration assistance device and the second housing part being designed as a contact area that is airtight during operation.

In such an embodiment, the whole system of housing and respiration assistance device can be easily sealed off in the circumferential contact area. The air flowing out of the respiration assistance device during operation can flow past gaps provided between the first and second housing parts and thus reach the side of the first housing part directed away from the respiration assistance device, where the at least one medium reservoir is preferably arranged. Embodiments are particularly preferred in which the two housing parts are designed to be displaceable relative to each other in the main direction of orientation of the outlet channel and in which a relative displacement takes place in the course of actuation, as a result of which a protective foil covering the medium from the outside is opened.

In a development of the invention, releasable activation-blocking means are provided on the housing which, when the housing is joined to the respiration assistance device, prevent the first housing part and the second housing part from moving relative to each other.

These activation-blocking means ensure that the pushing on and securing of the respiration assistance device onto the housing does not lead to opening of the medium reservoir preferably closed by means of a foil. In this way it is possible to provide the housing or respiration assistance device with securing means which are transferred to a secured state only when a considerable force is applied, without this leading to accidental dispensing of the medium. Such activation-blocking means can be formed, for example, by removable circumferential safety strips between the housing parts, by safety webs for breaking open, or also by bayonet mechanisms.

In a development of the invention, the housing is produced by injection molding and/or the bellows is produced by injection blow molding.

These production methods represent particularly inexpensive and reliable production methods.

### BRIEF DESCRIPTION OF THE DRAWINGS

Further advantages and features of the invention will become clear from the claims and from the following description of a preferred illustrative embodiment of the invention with reference to the drawings, in which:

FIG. 1 shows an inhaler housing in a non-activated state, without the respiration assistance device,

FIGS. 2a and 2b show a respiration assistance device with a bellows in a front view and a plan view,

FIG. 3 shows the complete inhalation device assembled from the housing of FIG. 1 and from the respiration assistance device of FIGS. 2a and 2b,

FIG. 4 shows the inhalation device in an activated state ready for use, and

FIG. 5 shows the inhalation device during the dispensing procedure.

### DETAILED DESCRIPTION OF THE ILLUSTRATIVE EMBODIMENTS

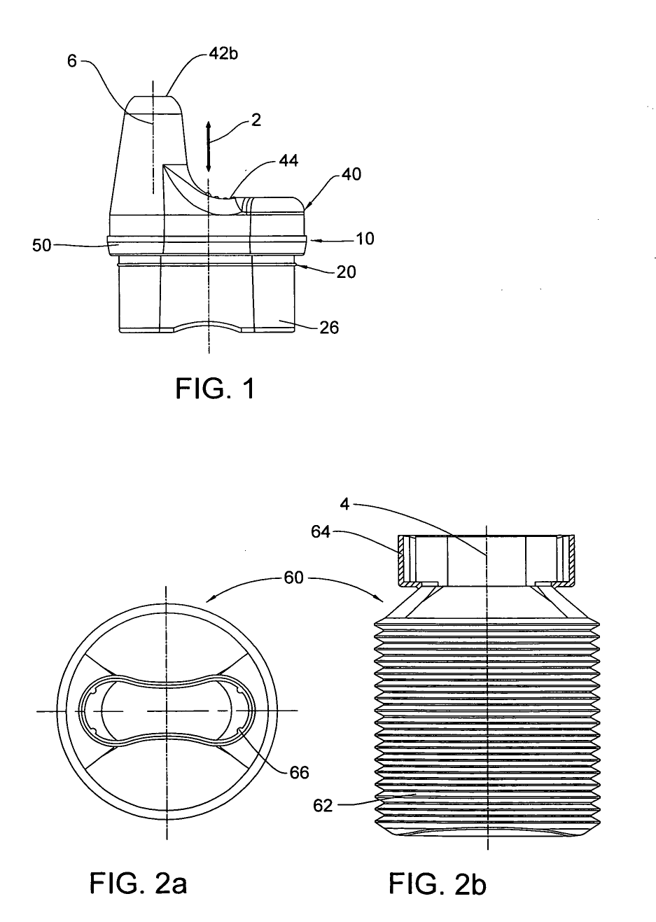

FIG. 1 shows a housing 10 of an inhalation device according to the invention. This housing 10 is composed of a first housing part 20 in which, in a manner not shown in FIG. 1, two medium reservoirs 24 are provided, which are each filled with a powdery medium 30. In a contact area at the lower end, the first housing part 20 is designed as a cone stump 26. A second housing part 40 is fitted onto the first housing part 20 and has an outlet channel 42 and a finger support 44 on the side directed away from the first housing part 20. In the state shown, which corresponds to the state in which the housing 10 is supplied, the two housing parts 20, 40 are connected to each other by means of a securing ring 50 that simultaneously serves as a tamper-evident closure, and they are specifically connected to each other such that they cannot move relative to each other in the direction of a main axis 2 of the housing 10 before the securing ring 50 is removed.

The second component of the inhalation device according to the invention described here is the respiration assistance device 60, which is shown in a front view and a plan view in FIGS. 2a and 2b. It comprises a bellows 62, which forms a compression chamber 62. At the upper end, the bellows 62 is adjoined by a cylindrical connecting portion 64. The whole respiration assistance device 60 is produced in one piece from an airtight and elastic material, preferably polyethylene. A total of four tensioning ribs 66 are provided on the inner face of the connecting portion 64 and are designed as local thickened areas of the connecting portion 64.

FIG. 3 shows the housing 10 of the inhalation device and also the respiration assistance device 60 in an assembled state, the respiration assistance device 60 being shown in cross section in the area of the connecting portion 64. The housing 10 is still in the same state as in FIG. 1, secured by the securing portion 50. The elastically designed connecting portion 64 is pushed onto the conical contact area 26 of the first housing part 20 of the housing 10. While it is being pushed on, the securing portion 50 ensures that the two housing parts 20, 40 do not move relative to each other. The cone stump 26 of the first housing part 20 and the connecting portion 64 are dimensioned such that the connecting portion 64 is elastically tensioned tangentially, with the result that a force-fit connection arises between the respiration assistance device 60 and the housing 10.

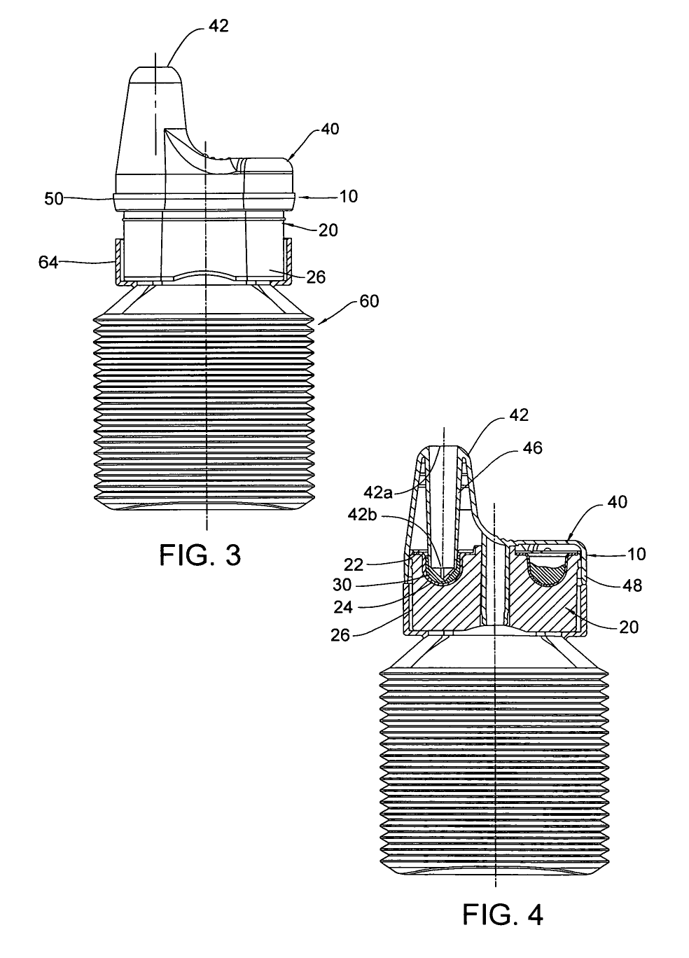

FIG. 4 shows a partially sectional view of the inhalation device after removal of the securing ring 50 and after activation, effected by pushing the two housing parts 20, 40 into each other. In the course of the second housing part 40 being pushed into the first housing part 20, an inner channel portion 46 of the outlet channel 42 is forced through a protective foil 22 into one of the two medium reservoirs 24 and the latter is thus opened. The second housing part 40 is pushed onto the first housing part 20 until an annular contact area 48 of the second housing part 40 bears on the annular end face of the connecting portion 64 of the respiration assistance device 60.

In this activated state, the user brings the inhalation appliance to his nose and inserts the outlet channel 42 into a nostril, the outer wall of the outlet channel 42 ending flush with the nostril, and inhalation through this nostril being possible only via the outlet channel 42. To inhale the medium 30, the user closes the other nostril with one hand and then uses his other hand to actuate the inhalation device while at the same time breathing in through the nose. The actuation is effected by compressing the bellows 62 in a direction of actuation 4. This is done by pressing together the index finger of the actuating hand resting on the finger support 44 and the thumb resting on the underside 68 of the bellows 62. By virtue of the parallel orientation of the main direction of orientation 6 of the outlet channel 42 and of the direction of actuation 4 of the bellows 62 and their small distance from each other, there is no fear of the device slipping out of the nostril. A slight and insignificant displacement of the device in the main direction of orientation 6 of the outlet channel 42 may at most result from the actuation. This, however, is not problematic with respect to successful inhalation.

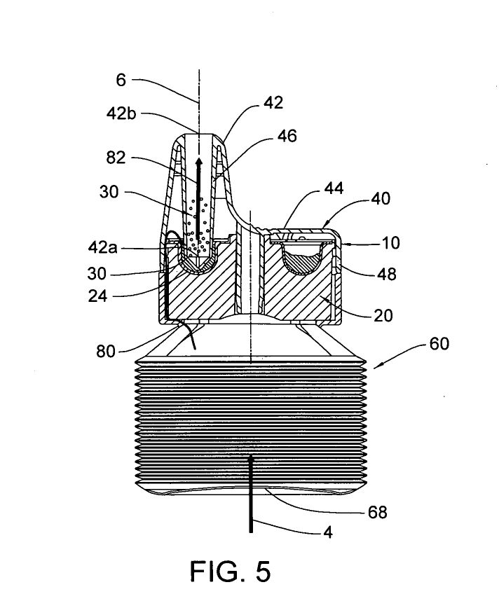

As is shown in FIG. 5, the air located beforehand in the bellows 62 escapes from the bellows 62 along the arrow direction 80 into the housing 10. It flows into the medium reservoir 24 and blows the powdery medium 30 located in the latter into the intake side 42a of the outlet channel 42. At the same time, the air is sucked out through the output side 42b by means of the inhalation performed by the user, along the path indicated by the arrow 82 into the user's nose. The overpressure that the air from the bellows 62 causes in the housing 10, and the underpressure in the outlet channel 42 caused by the user breathing in, together lead to complete removal of the powdery medium 30 through the inner channel portion 46 of the outlet channel 42 and into the user's nose.

Even if the outlet channel 42 does not lie flush on the inner face of the user's nostril, and even if the user is too weak or short of breath to be able to breathe in sufficiently strongly throughout the inhalation process, the overpressure in the housing 10 still ensures complete removal of the powdery medium.

After the powdery medium from the medium reservoirs has been used up, the bellows can be withdrawn again from the housing and can then be fitted onto a new, unused housing of an inhalation appliance.

Without any design changes, the housing 10 of the inhalation device can also be used without the bellows 62, such that a high degree of flexibility is obtained. Users who have difficulty with the inhalation use the inhalation appliance in conjunction with the bellows 62, while users who have no problems without the bellows 62 can dispense with using the latter.

In other embodiments not shown, the housing has only one medium reservoir or has more than two medium reservoirs.

The invention likewise encompasses embodiments (not shown) in which the flow path of the air from the respiration assistance device into the housing of the inhalation device does not pass through grooves or tensioning ribs in the contact area between the components, but is instead provided as a separate air inlet preferably centrally on an underside of the housing.

Other respiration assistance devices, particularly with electrically operated pumps, can also be used in further embodiments of the inhalation device according to the invention.

1. Inhalation device for an in particular powdery medium (30), with a housing (10) having
at least one medium reservoir (24) and
an outlet channel (42),
in which a first end (42a) of the outlet channel (42) opens out in the area of the medium reservoir (24) and a second end (42b) is designed to lie flush on a respiration opening of a user, such that the medium (30) stored in the medium reservoir (24) can be inhaled by the user breathing in,
characterized by
a respiration assistance device (60) which can be connected releasably to the housing (10) and from which an air stream assisting the inhalation can be delivered to the housing (10) by means of a manual actuation.

2. Inhalation device according to claim 1, characterized in that the respiration assistance device (60) is designed for a manual actuation in a direction of actuation (4) which is parallel to a main direction of orientation (6) of the outlet channel (42).

3. Inhalation device according to claim 1, characterized in that the respiration assistance device (60) has a compression chamber (62) whose volume can be mechanically reduced directly by the manual actuation.

4. Inhalation device according to claim 3, characterized in that the respiration assistance device (60) has a bellows (62).

5. Inhalation device according to claim 1, characterized in that a finger support surface (44) is provided on a side of the housing (10) directed away from the respiration assistance device (60).

6. Inhalation device according to claim 1, characterized in that the respiration assistance device (60) and the housing (10) are designed in a contact area in such a way that a force applied by the manual actuation leads to portions (48, 64) arranged on the housing and on the respiration assistance device being pressed tightly onto each other in the contact area.

7. Inhalation device according to claim 1, characterized in that, in a contact area, the respiration assistance device (60) and the housing (10) have sealing and/or securing means (26, 64, 66) acting with a force fit and/or a form fit.

8. Inhalation device according to claim 7, characterized in that the securing means comprise a push-on cone (26) on the housing and a push-on portion (64, 66) on the respiration assistance device.

9. Inhalation device according to claim 1, characterized in that the housing (10) is designed in several parts, in particular in two parts, the at least one medium reservoir (24) being designed in a first housing part (20), and the outlet channel (42) being designed on a second housing part (40), and in that a circumferential contact area between the respiration assistance device (60) and the second housing part (40) is designed as a contact area that is airtight to the outside during operation.

10. Inhalation device according to claim 8, characterized in that releasable activation-blocking means (50) are provided on the housing (10) and, when the housing (10) is joined to the respiration assistance device (60), prevent the first housing part (20) and the second housing part (40) from moving relative to each other.

11. Inhalation device according to claim 1, characterized in that the housing is produced by injection molding and/or the bellows is produced by injection blow molding.
